(19) United States
(12) Patent Application Publication
(10) Pub. No.: US 2026/0311223 A1
(43) Pub. Date: Oct. 8, 2026

(54) OPHTHALMOLOGY BILLING CODE ELIGIBILITY AND REIMBURSEMENT RANKING SYSTEM

(71) Applicant: OCO 2020 INC., Austin, TX (US)

(72) Inventor: Johnathan Jeffers, Austin, TX (US)

(73) Assignee: OCO 2020 INC., Austin, TX (US)

(21) Appl. No.: 19/637,388

(22) Filed: Apr. 2, 2026

Related U.S. Application Data
(60) Provisional application No. 63/782,781, filed on Apr. 3, 2025.

(51) Int. Cl. G06Q 30/04; G06Q 40/08; G16H 10/60

(52) U.S. Cl. CPC G06Q 30/04; G06Q 40/084; G16H 10/60

(57) ABSTRACT

Apparatus and associated methods relate to ophthalmology billing code selection. In an illustrative example, a system may receive patient insurance data and exam details, may query a database of billing codes and reimbursement values, and may generate a billing code recommendation. For example, the system may compare reimbursement values for multiple applicable codes and recommend the highest-value option. Various embodiments may advantageously increase billing efficiency for ophthalmology practices by automating code selection processes that account for differences between private insurance and Medicare reimbursement structures. Various embodiments may achieve one or more advantages. For example, some embodiments may reduce administrative burden on physicians, minimize coding errors, and ensure compliant billing practices. The automated system may adapt to changes in reimbursement policies, which in turn may allow practices to maintain optimal billing without frequent manual updates. Additionally, the integration with electronic medical record systems can streamline workflow, thereby improving efficiency.

### CROSS-REFERENCE TO RELATED APPLICATIONS

This application also claims the benefit of U.S. Provisional Application Ser. No. 63/782,781, titled “OPHTHALMOLOGY BILLING CODE SELECTION SYSTEM,” filed by Johnathan Jeffers on Apr. 3, 2025.

### TECHNICAL FIELD

Various embodiments relate generally to medical billing systems, and more particularly to an ophthalmology billing code generation system for presenting billing codes based on patient information, insurance data, and/or exam details.

### BACKGROUND

Medical billing systems often play a role in the healthcare industry, facilitating the complex process of submitting and processing claims for reimbursement. These systems have evolved over time to accommodate the increasing complexity of healthcare services and insurance policies. In the field of ophthalmology, billing systems often handle a range of procedures and treatments specific to eye care.

Healthcare providers often utilize various methods to manage their billing processes. These methods tend to range from manual data entry to software solutions. Additionally, these systems typically involve capturing patient information, documenting procedures, assigning billing codes, and submitting claims to insurance companies. Some systems integrate with electronic medical records (EMR) to streamline the flow of information between clinical and administrative functions.

In ophthalmology practices, billing systems often handle unique coding requirements. For example, ophthalmologists may use both evaluation and management (E/M) codes as well as ophthalmology specific eye-codes for different types of examinations and procedures. The selection of codes can impact reimbursement rates and compliance with insurance policies. As healthcare regulations and reimbursement models continue to change, ophthalmology billing systems may adapt to developing requirements and select code selection processes to ensure accurate billing practices.

### SUMMARY

Apparatus and associated methods relate to ophthalmology billing code selection. In an illustrative example, a system may receive patient insurance data and exam details, may query a database of billing codes and reimbursement values, and may generate a billing code recommendation. For example, the system may compare reimbursement values for multiple applicable codes and recommend the highest-value option. Various embodiments may advantageously increase billing efficiency for ophthalmology practices by automating code selection processes that account for differences between private insurance and Medicare reimbursement structures.

Various embodiments may achieve one or more advantages. For example, some embodiments may reduce administrative burden on physicians, minimize coding errors, and ensure compliant billing practices. The automated system may adapt to changes in reimbursement policies, which in turn may allow practices to maintain optimal billing without frequent manual updates. Additionally, the integration with electronic medical record systems can streamline workflow, thereby improving practice efficiency.

The details of various embodiments are set forth in the accompanying drawings and the description below. Other features and advantages will be apparent from the description and drawings, and from the claims.

### BRIEF DESCRIPTION OF THE DRAWINGS

FIG. 1 illustrates a block diagram of a medical billing system employed in an illustrative use-case scenario.

FIG. 2 illustrates a block diagram of a medical billing system architecture.

FIG. 3 illustrates a block diagram of a data processing system for analyzing medical billing information.

FIG. 4 illustrates a flowchart of a medical billing code selection process.

FIG. 5 illustrates a flowchart of a medical billing code selection process.

FIG. 6 illustrates a flowchart of a code selection process for medical billing.

FIG. 7 illustrates a flowchart of an exemplary eligibility-gated billing code selection workflow.

FIG. 8 illustrates a flowchart of exemplary eligibility rule evaluation engines.

FIG. 9 illustrates exemplary structured data objects used by the billing code selection engine.

Like reference symbols in the various drawings indicate like elements.

### DETAILED DESCRIPTION OF ILLUSTRATIVE EMBODIMENTS

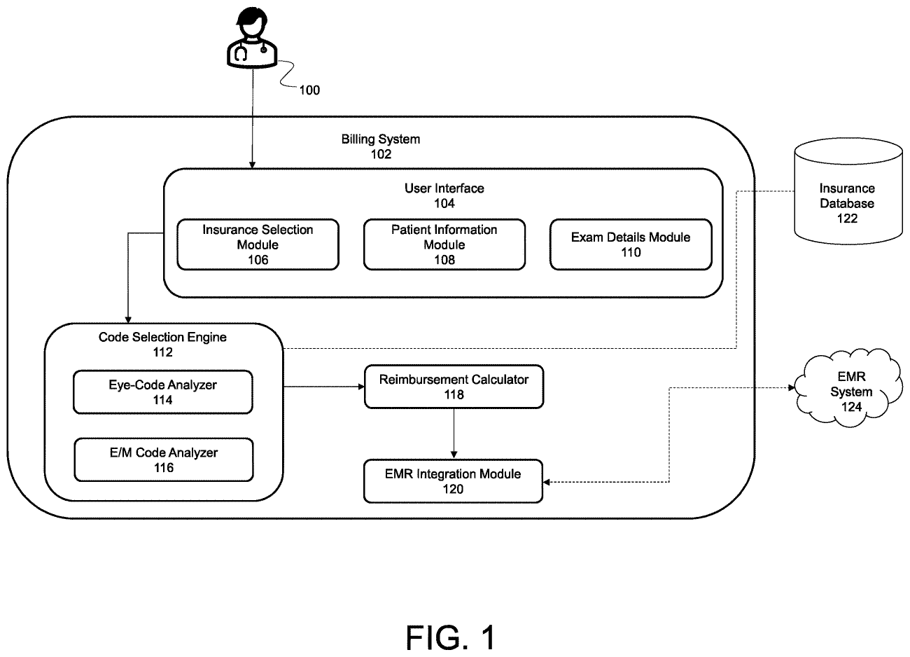

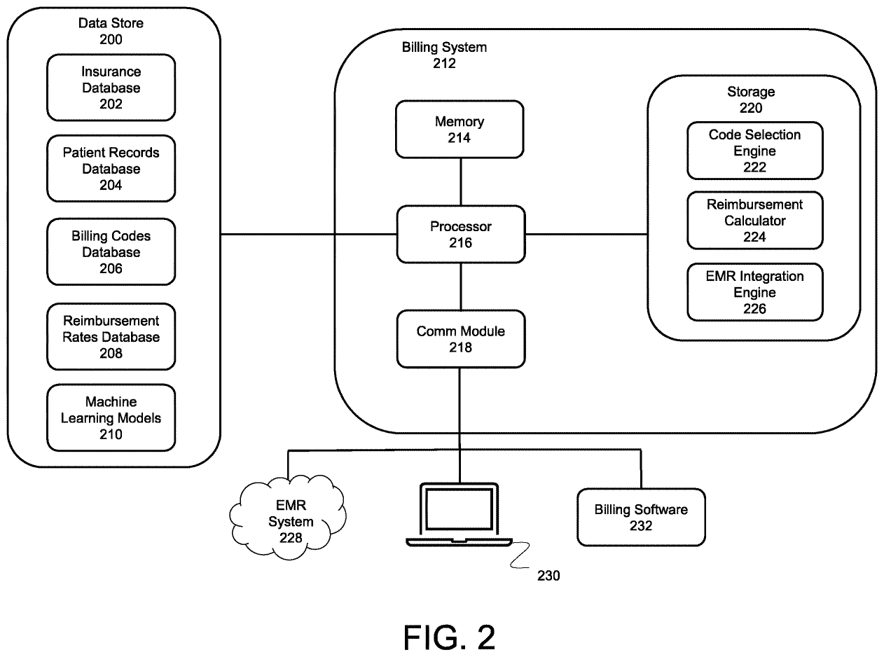

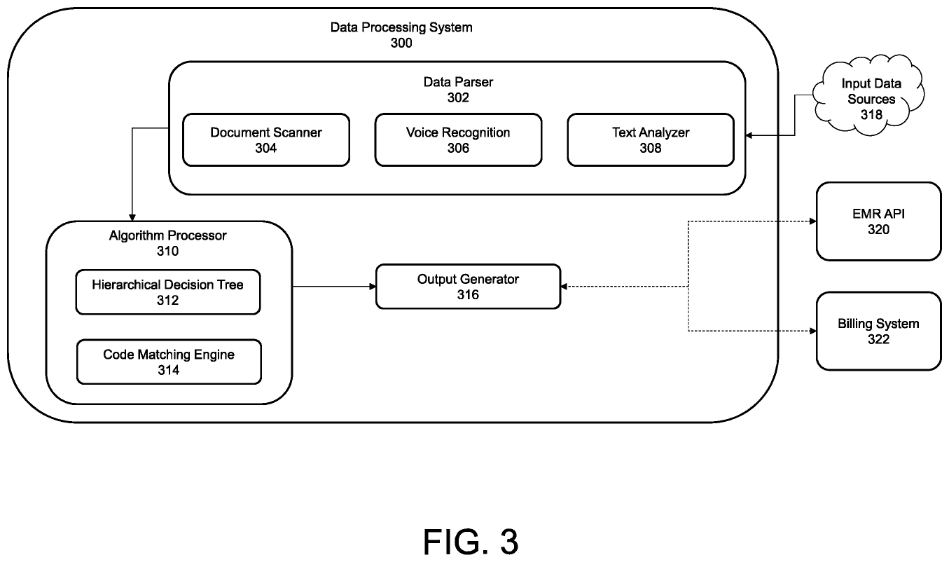

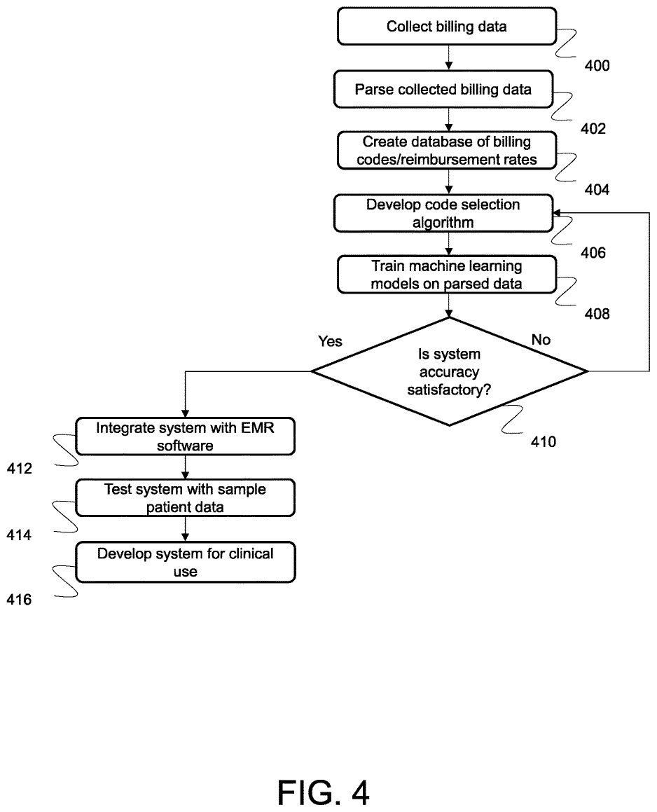

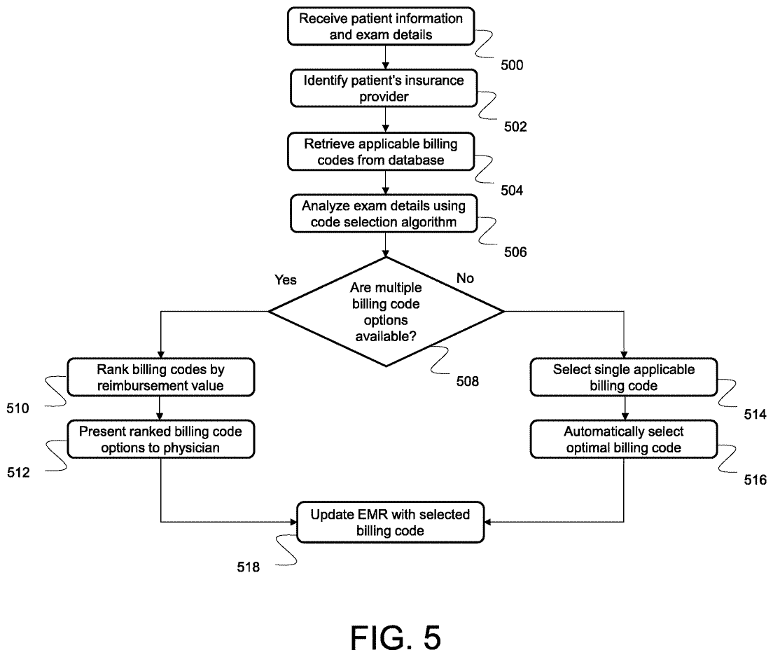

To aid understanding, this document is organized as follows. First, to help introduce discussion of various embodiments, a medical billing code selection system is introduced with reference to FIGS. 1 and 2. Second, that introduction leads into a description with reference to FIGS. 3-5 of some exemplary embodiments of the medical billing code selection system. Third, with reference to FIGS. 2-6, the medical billing code selection system is described in application to exemplary ophthalmology billing scenarios. Fourth, with reference to FIGS. 1-3 the discussion turns to exemplary embodiments that illustrate how the medical billing code selection system can be developed and implemented. Fifth, and with reference to FIGS. 1-6, this document describes exemplary apparatus and methods useful for medical billing code selection. Sixth, this disclosure turns to a review of experimental data and a discussion of efficiency improvements enabled by the medical billing code selection system. One topology is reviewed with reference to FIG. 4. A second exemplary embodiment of the medical billing code selection system is reviewed with reference to FIG. 5. Seventh, the document introduces details of the code selection algorithms and machine learning models. Finally, the document discusses further embodiments, exemplary applications and aspects relating to integration with electronic medical record systems and billing software.

In some examples, the medical billing code selection system (MBCOS) may provide an approach to challenges in selecting appropriate billing codes for ophthalmology practices. For example, the MBCOS may utilize advanced data processing techniques to analyze patient information, insurance details, and exam details to recommend optimal billing codes. In some examples, the MBCOS may transform raw input data through multiple stages of processing. In some implementations, the MBCOS may parse diverse data sources including but not limited to scanned documents, voice input, text entries, and/or the like. This parsed data may, for example, then be processed through specialized algorithms that apply hierarchical decision trees and code matching engines. The algorithms may consider the unique aspects of ophthalmology billing, including the use of both evaluation and management (E/M) codes and ophthalmology-specific codes.

In some examples, components of the system may include a data parser for interpreting various input formats, an algorithm processor for code selection, and an output generator that interfaces with EMR systems and billing software. Additionally, the system may incorporate machine learning models that continuously improve code selection accuracy based on historical data and outcomes.

In some examples, the billing system may be specifically designed for ophthalmology practices. This specialization may allow the system to address the unique billing requirements and code structures used in ophthalmology, such as the distinction between E/M codes and ophthalmology-specific codes. The system's algorithms may be tailored to understand the nuances of ophthalmological exams and procedures, thereby enabling more accurate code selection compared to general medical billing systems.

In some examples, by automating the code selection process, the system may advantageously reduce the burden on healthcare providers, minimize billing errors, and/or maximize appropriate reimbursement. The approach provided by this system may transform the billing process from a manual task to a data-driven operation that integrates with existing healthcare IT infrastructure.

FIG. 1 illustrates a block diagram of a medical billing system (MBS) employed in an illustrative use-case scenario. In traditional methods, medical providers often struggle with manually looking up codes in printed materials or basic electronic databases, which can be time-consuming and prone to errors. This MBS may transform the billing process by automating code selection.

In some examples, a medical provider 100 may interact with the billing system 102 through a user interface 104. As shown, the user interface 104 includes an insurance selection module 106, a patient information module 108, and an exam details module 110. These modules may allow the medical provider 100 to input relevant data efficiently, which in turn addresses the issue of scattered information in traditional billing methods.

In some examples, the billing system 102 includes a code selection engine 112 that processes the input data to determine appropriate billing codes. As shown, the code selection engine 112 contains an Eye-code analyzer 114 for analyzing ophthalmology-specific billing codes and an E/M code analyzer 116 for analyzing evaluation and management codes. This dual-analyzer approach may address the challenge in ophthalmology billing where multiple sets of codes may apply.

In some examples, a reimbursement calculator 118 within the billing system 102 may receive input from the code selection engine 112 to determine reimbursement values. This feature may help medical providers understand the financial implications of different code selections, thereby addressing the issue of potential under-billing in traditional methods.

As shown, the billing system 102 includes an EMR integration module 120 that facilitates communication with an EMR system 124. This integration may streamline the billing process by automatically updating patient records with billing information, thereby reducing the risk of data entry errors common in manual processes.

In some examples, an insurance database 122 connected to the billing system 102 may store up-to-date insurance information and reimbursement rates. This database may ensure that the code selection and reimbursement calculations are based on current information, thereby addressing the challenge of outdated rate information in traditional billing methods.

In some examples, the billing system 102 may include a data parser that can take inputs from scanned documents, voice, text, images, and/or the like. This feature may allow for flexible data input methods, thereby addressing the challenge of rigid data entry systems in traditional billing processes. The billing system 102 may include Optical Character Recognition (OCR) capability to process scanned documents, which in turn may further enhance the system's ability to handle diverse input formats.

In some examples, by transforming raw input data through multiple stages of processing, including parsing, analysis, and code matching, the billing system 102 may provide a comprehensive approach to the challenges of medical billing. In various examples, the billing system 102 may be configured to advantageously process diverse input formats, analyze complex coding requirements, and/or integrate with existing EMR systems.

FIG. 2 illustrates a block diagram of a medical billing system architecture. As shown, the architecture includes a data store 200, a billing system 212, and external components that work together to process billing information and generate optimal code selections. For example, the billing system 102 may include the billing system 212.

In some examples, the data store 200 may contain multiple databases that provide information for the billing process. The data store 200 may include an insurance database 202, a patient records database 204, a billing codes database 206, and a reimbursement rates database 208. The insurance database 202 may store information about various insurance providers and their policies. The patient records database 204 may contain medical histories and demographic information of patients. The billing codes database 206 may store a comprehensive list of medical billing codes, including both Eye-codes and E/M codes. The reimbursement rates database 208 may contain up-to-date information on reimbursement rates for different procedures and insurance providers.

In some examples, the data store 200 may include one or more machine learning models 210. These machine learning model(s) 210 may be trained on historical billing data to improve the accuracy of code selection over time. By incorporating machine learning, the system may adapt to changes in billing practices and billing code selection based on past outcomes.

In some examples, the billing system 212 may serve as the central processing unit for the medical billing architecture. In some examples, the billing system 212 includes memory 214, a processor 216, and a communication module 218. The memory 214 may store instructions and temporary data for processing. The processor 216 may execute the instructions and perform calculations for code selection and reimbursement calculation. The communication module 218 may facilitate data exchange between the billing system 212 and external components.

Connected to the billing system 212 is storage 220, which may contain specialized software components. These components may include a code selection engine 222, a reimbursement calculator 224, and an EMR integration engine 226. The code selection engine 222, for example, may analyze patient information, insurance details, and exam specifics to recommend appropriate billing codes. For example, the reimbursement calculator 224 may determine potential reimbursement amounts based on the selected codes and insurance information. The EMR integration engine 226 may facilitate seamless data exchange between the billing system 212 and external EMR systems.

The billing system 212 may, in some implementations, interact with several external components through the communication module 218. These external components may include an EMR system 228, a client device 230, and billing software 232. The EMR system 228 may store and manage patient medical records. The client device 230 may provide an interface for medical providers to interact with the billing system 212. The billing software 232 may process the selected codes and generate billing documentation.

In some examples, the billing system 212 can be implemented as a cloud-based system, for example, on Amazon Web Services or a similar cloud-based platform. This implementation may allow for scalability, easy updates, and remote access for multiple users. The cloud-based system may facilitate real-time updates to the databases in the data store 200, thereby ensuring that current information is available for code selection and reimbursement calculations.

In some examples, the billing system 212 can be implemented as a locally-run desktop or mobile application. This implementation may be useful for medical practices that prefer to keep their data on-premises or have limited internet connectivity. For example, the locally-run application may periodically sync with a central database to ensure up-to-date information while maintaining local processing capabilities.

The architecture described in FIG. 2 may represent an approach to medical billing by integrating multiple data sources, advanced processing capabilities, and machine learning models into a cohesive system. This integration may allow for more accurate code selection, efficient reimbursement calculations, and seamless integration with existing medical record systems. The flexibility of implementation, either as a cloud-based system or a local application, may further enhance the system's adaptability to various healthcare settings.

FIG. 3 illustrates a block diagram of a data processing system 300 for analyzing medical billing information. As shown, the data processing system 300 includes a data parser 302, an algorithm processor 310, and an output generator 316 that work together to process various types of input data and generate billing code recommendations.

In some examples, the data parser 302 may receive information from input data sources 318. For example, the input data sources 318 may include an external EHR system (e.g., through application programming interfaces (APIs) including FHIR (Fast Healthcare Interoperability Resources) and HL7 APIs, Cerner Ignite APIs, athenahealth Marketplace APIs). The data parser 302 may contain multiple analysis components for processing different types of input data. These components may include a document scanner 304, voice recognition 306, and text analyzer 308. The document scanner 304 may process scanned documents and images, extracting relevant billing information. The voice recognition 306 may convert spoken instructions or dictated notes into text data. The text analyzer 308 may process written notes, reports, or other text-based inputs. By incorporating these diverse input processing capabilities, the data parser 302 may allow for flexible data entry methods, thereby addressing the challenge of handling various input formats in medical billing.

In some examples, the data parser 302 may communicate with the algorithm processor 310, which may contain a hierarchical decision tree 312 and a code matching engine 314. The hierarchical decision tree 312 may use a series of if-then statements to navigate through the complex decision-making process of selecting appropriate billing codes. This approach may allow for a structured and logical progression through the various factors that influence code selection, such as patient information, exam details, and insurance requirements.

In some examples, the code matching engine 314 within the algorithm processor 310 may compare the processed input data against a database of billing codes to identify the most appropriate matches. In some examples, the code matching engine 314 may utilize machine learning algorithms to improve its accuracy over time based on historical data.

In some examples, the algorithm processor 310 may connect to the output generator 316, which may produce billing code recommendations based on the processed data. The output generator 316 may format the selected codes and associated information in a way that is compatible with various downstream systems.

In some examples, the output generator 316 can communicate with an EMR API 320 and a billing system 322. The EMR API 320 may allow for seamless integration of the billing code recommendations into the patient's electronic medical record, thereby reducing the practice of manual data entry and potential errors. The billing system 322 may use the generated codes to create accurate billing statements and insurance claims.

In some examples, the data processing system 300 may transform raw input data through multiple stages of processing, ultimately providing structured output to the EMR API 320 and billing system 322. This multi-stage processing approach allows for more accurate and efficient billing code selection compared to traditional manual methods.

In some examples, the code selection engine may use the hierarchical decision tree 312 to select billing codes. The hierarchical structure may allow for a systematic evaluation of various factors that influence code selection, such as the type of exam performed, the complexity of the patient's condition, the specific procedures carried out, and/or the like. By using this structured approach, the code selection engine may navigate the complex landscape of medical billing codes, particularly in specialized fields like ophthalmology where multiple code sets may apply.

In some examples, the billing system 322 may connect to insurance company databases to retrieve up-to-date coding criteria. This connection may allow the system to access the most current information on billing requirements and reimbursement rates. By maintaining a direct link to insurance company databases, for example, the billing system 322 may ensure that code selections are based on the latest guidelines. For example, the connection between the billing system 322 and the insurance company database may advantageously reduce the risk of claim rejections due to outdated information. For example, the billing system 322 may advantageously keep up with frequent changes in insurance policies and coding requirements.

FIG. 4 illustrates a flowchart of a medical billing code selection process. The process begins at a step 400 with collecting billing data. This step may involve gathering information from various sources, including but not limited to patient records, insurance claims, and medical procedure documentation. Collecting comprehensive billing data may be useful for establishing a robust foundation for the medical billing code selection process.

At step 402, the collected billing data may be parsed. For example, the data parser 302 may extract relevant details (e.g., procedure codes, diagnosis codes, reimbursement amounts) and transform raw billing information into a structured format. For example, the structured data may be advantageously more efficient to be analyzed by subsequent steps in the process.

At step 404, a database of billing codes and reimbursement rates may be created. This step may involve populating the billing codes database 206 and reimbursement rates database 208 with the parsed information. Creating these databases may be useful as it provides a centralized repository of up-to-date billing information that can be quickly accessed and updated.

At step 406, a code selection algorithm may be developed. For example, the algorithm processor 310 may incorporate the hierarchical decision tree 312 and code matching engine 314. Developing a specialized code selection algorithm may be useful as it allows for tailored decision-making based on the specific requirements of medical billing, particularly in specialized fields like ophthalmology.

At step 408, the machine learning models may be trained on the parsed data. For example, the machine learning models 210 may be learned from historical billing data to improve code selection accuracy. Training machine learning models may be useful as it allows the system to adapt to changing billing patterns and improve its performance over time.

At step 410, the system may evaluate whether the system accuracy is satisfactory. This step may involve testing the trained models against a set of known billing scenarios to assess their performance. If the accuracy is not satisfactory, the process may return to step 408 to continue training the machine learning models. This iterative approach may be useful as it ensures continuous improvement of the system's accuracy.

If the system accuracy is satisfactory, the process may advance to step 412, where the system may be integrated with EMR software. The EMR integration engine 226 may, for example, facilitate seamless data exchange between the billing system 212 and the EMR system 228. Integration with EMR software may be useful as it allows for real-time access to patient information and streamlines the billing process.

At step 414, the system may be tested with sample patient data. This step may involve running the billing code selection process through a series of test cases to ensure its functionality in real-world scenarios. Testing with sample data may be useful as it allows for adjustments and validation of the system's performance before clinical deployment.

At step 416, the system may be developed for clinical use. This step may involve preparing user interfaces, documentation, and training materials for medical providers. Developing the system for clinical use may be useful as it ensures that the billing system may be effectively implemented in healthcare settings.

The medical billing code selection process illustrated in FIG. 4 may represent a comprehensive approach to improving billing accuracy and efficiency. By incorporating data collection, machine learning, and integration with existing healthcare systems, this process may address many of the challenges associated with traditional medical billing methods.

FIG. 5 illustrates a flowchart of a medical billing code selection process. The process begins at step 500 with receiving patient information and exam details. This step may involve the medical provider 100 inputting data through the user interface 104 of the billing system 102. Receiving comprehensive patient information and exam details may be useful as it provides context for accurate code selection.

At step 502, the process may identify the patient's insurance provider. For example, the insurance database 122 may be queried to retrieve relevant insurance information. Identifying the insurance provider may be useful as it allows the system to consider specific reimbursement rates and coding requirements for each insurer.

At step 504, applicable billing codes may be retrieved from a database. The billing codes database 206 may be accessed to fetch relevant codes based on the exam details and insurance provider, for example. Retrieving applicable codes may be useful as it narrows down the potential options for billing.

At step 506, the process may analyze exam details using a code selection algorithm. The code selection engine 222 may utilize the hierarchical decision tree 312 and the code matching engine 314 to process the exam information. Analyzing exam details with a specialized algorithm may be useful as it allows for nuanced interpretation of medical procedures, particularly in specialized fields like ophthalmology.

At step 508, a determination is made whether multiple billing code options are available. This decision point may be useful as it allows the system to handle both straightforward and complex billing scenarios.

If multiple options are available, the process moves to step 510 to rank billing codes by reimbursement value. The reimbursement calculator 224 may be used to determine the potential reimbursement for each code option. Ranking codes by reimbursement value may be useful as it provides the medical provider with an understanding of the financial implications of different code selections.

At step 512, the ranked billing code options may be presented. This presentation may occur through the user interface 104, allowing the medical provider to make an informed decision. Presenting ranked options may be useful as it empowers the medical provider to choose the most appropriate code while considering reimbursement potential.

If multiple billing code options are not available, the process moves to step 514 to select a single applicable billing code. This step may ensure that even in straightforward cases, an appropriate code is chosen.

At step 516, the optimal billing code may automatically be selected. This automatic selection may be based on predefined criteria or machine learning models 210 trained on historical data. Automatic selection of the optimal code may be useful as it reduces the burden on the medical provider and improves consistency in billing practices.

At step 518, the EMR system 228 may be updated with the selected billing code. The EMR integration engine 226 may facilitate this update, thereby ensuring that the patient's medical record reflects the most current billing information. Updating the EMR may be useful as it maintains a comprehensive and accurate record of the patient's care and associated billing.

The medical billing code selection process illustrated in FIG. 5 may represent a systematic approach to billing code selection. In some examples, by incorporating patient information, insurance details, and/or exam specifics, the process may advantageously address the complexities of medical billing, particularly in specialized fields like ophthalmology. The inclusion of decision points and ranking mechanisms may allow for flexibility in handling various billing scenarios while providing valuable information to guide code selection.

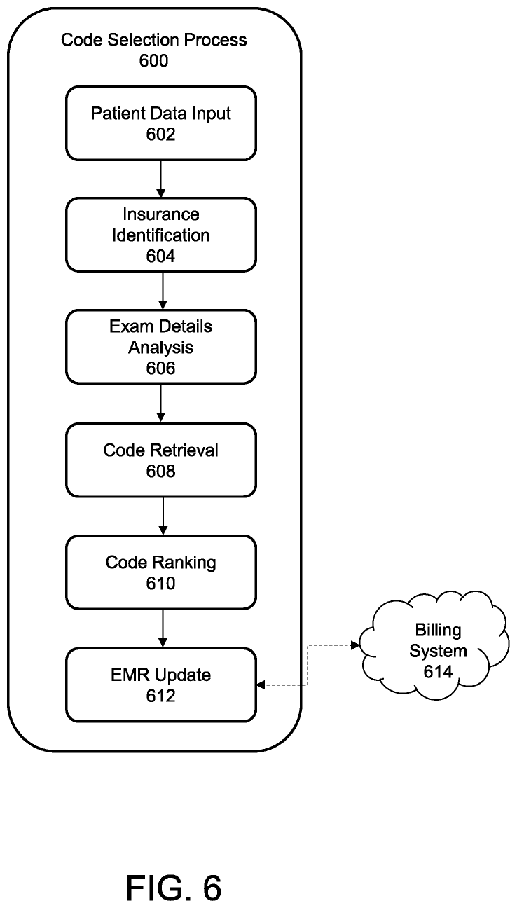

FIG. 6 illustrates a flowchart of an exemplary code selection process 600 for medical billing. The code selection process 600 begins with patient data input 602, where initial patient information is received. In some examples, the medical provider 100 may input this data through the user interface 104 of the billing system 102. The patient data input 602 may include demographic information, medical history, and current symptoms.

In some examples, following the patient data input 602, the code selection process 600 may move to insurance identification 604. During this step, the patient's insurance information may be determined. In some examples, the insurance database 122 may be queried to retrieve relevant insurance details. The insurance identification 604 may be useful as it allows the billing system 102 to consider specific reimbursement rates and coding requirements for each insurer. This step may be useful as it tailors the subsequent code selection process to the patient's specific insurance coverage.

In some examples, after insurance identification 604, the code selection process 600 may proceed to exam details analysis 606. In this step, information about the medical examination may be analyzed. The code selection engine 112 may utilize the Eye-code analyzer 114 and E/M code analyzer 116 to process the exam information. The exam details analysis 606 may be useful as it provides a comprehensive understanding of the medical services provided, which is helpful for accurate code selection. This step may be useful as it can handle the complexities of specialized fields like ophthalmology, where multiple code sets may apply.

In some examples, the code selection process 600 may continue to code retrieval 608. In this step, relevant billing codes may be retrieved based on the analyzed exam details. The billing codes database 206 may be accessed to fetch applicable codes. The code retrieval 608 may be useful as it narrows down the potential options for billing, thereby streamlining the selection process. This step may be useful as it can incorporate both general medical codes and specialty-specific codes, such as those used in ophthalmology.

In some examples, the code selection process 600 may then advance to code ranking 610. In this step, the retrieved codes may be ranked according to predetermined criteria. The reimbursement calculator 118 may be used to determine the potential reimbursement for each code option. The code ranking 610 may be useful as it provides a clear hierarchy of billing options, allowing for informed decision-making. This step may be useful as it considers both the appropriateness of the code for the medical service provided and the potential reimbursement value.

In some examples, following code ranking 610, the code selection process 600 may move to EMR update 612. In this step, the electronic medical record may be updated with the selected codes. The EMR integration engine 226 may facilitate this update, thereby ensuring that the patient's medical record reflects the most current billing information. The EMR update 612 may be useful as it maintains a comprehensive and accurate record of the patient's care and associated billing. This step may be useful as it integrates the billing process directly with the patient's medical record, improving overall data consistency and reducing the risk of billing errors.

In some examples, the code selection process 600 may include interaction with a billing system 614, which is shown connected to EMR update 612. The billing system 614 may process the selected codes for billing purposes. In some examples, the billing system 614 may generate billing invoices based on the selected codes and patient information. This feature may be useful as it automates the creation of billing documentation, reducing manual effort and potential errors.

In some examples, the billing system 614 may include an API (e.g., as described with reference to FIG. 3) to integrate with and update EMR systems. This API may allow for data exchange between the billing system 614 and external EMR systems. The integration may be useful as it ensures that billing information is consistently reflected across all relevant healthcare IT systems. This feature may be useful as it bridges the gap between billing processes and patient care documentation, thereby promoting a more holistic approach to healthcare information management.

The flowchart may show a sequential progression through the steps, with each step building upon the information gathered and processed in previous steps. This sequential nature may be useful as it ensures a logical approach to code selection. The process may be useful in its ability to handle complex billing scenarios while maintaining accuracy.

In some examples, by transforming raw patient data through multiple stages of analysis and decision-making, the code selection process 600 may represent a comprehensive approach to medical billing. The process may address many of the challenges associated with traditional billing methods, particularly in specialized fields like ophthalmology, by incorporating patient information, insurance details, and exam specifics into a streamlined, data-driven workflow.

In some examples, the medical billing code selection system may be adapted for use in various medical specialties beyond ophthalmology. The system's core functionality of analyzing patient data, insurance information, and procedure details to recommend optimal billing codes may be customized for specialties such as cardiology, orthopedics, or neurology. This adaptability may allow the system to address the specific billing requirements and code structures of different medical fields.

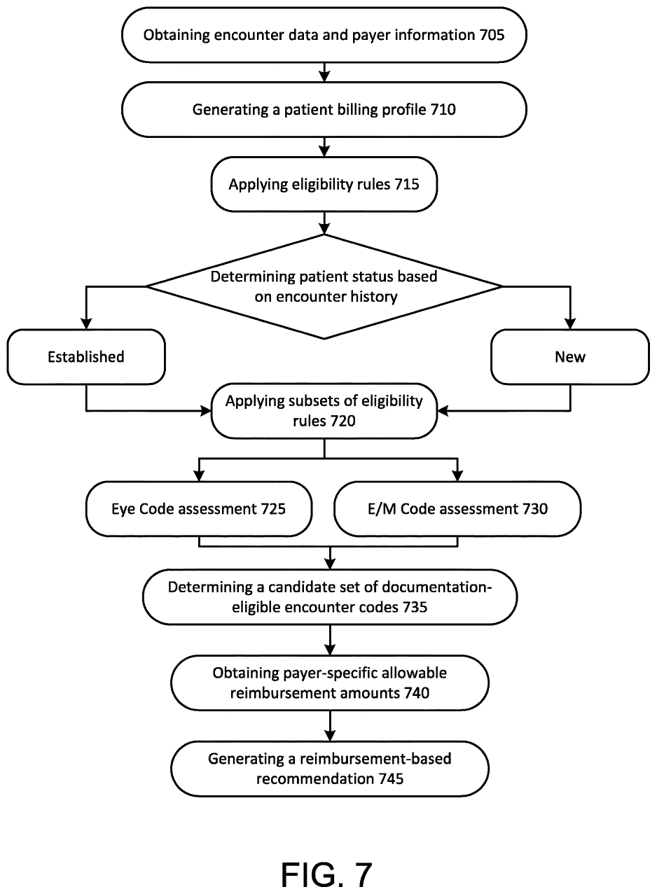

FIG. 7 illustrates an exemplary eligibility-gated billing code selection workflow for automatically generating a payer-specific reimbursement-based recommendation for an encounter. In some embodiments, the workflow of FIG. 7 may be implemented, for example, by the billing system 102 of FIG. 1, the billing system 212 of FIG. 2, and/or the data processing system 300 of FIG. 3. As shown, the workflow may transform ophthalmology encounter data and payer information into machine-usable intermediate data structures, may apply multiple subsets of eligibility rules to the intermediate data structures, may determine a constrained candidate set of eligible encounter codes, and may generate a reimbursement-based recommendation for presentation within an electronic medical record. In some embodiments, the workflow of FIG. 7 may provide a more technically structured approach than a generic code lookup process because reimbursement retrieval and ranking are performed only after eligibility has been computationally determined from normalized encounter attributes.

In some embodiments, the workflow may begin by obtaining encounter data and payer information at step 705. For example, the encounter data may be obtained from an EMR system 124 through an EMR integration module 120 and/or through an EMR API 320, as described with reference to FIGS. 1-3. In some embodiments, the obtained encounter data may include structured fields, semi-structured fields, and/or unstructured fields, such as exam documentation, chart notes, encounter timing information, prior encounter history, insurance identifiers, diagnostic impressions, treatment-plan information, and/or other clinical content. For example, as described with reference to FIG. 3, the data parser 302 may normalize heterogeneous source data into discrete attributes usable by downstream rule engines. In some embodiments, obtaining the encounter data and payer information at step 705 may include electronically retrieving, extracting, parsing, normalizing, and/or formatting the data into a machine-usable representation for subsequent computational analysis.

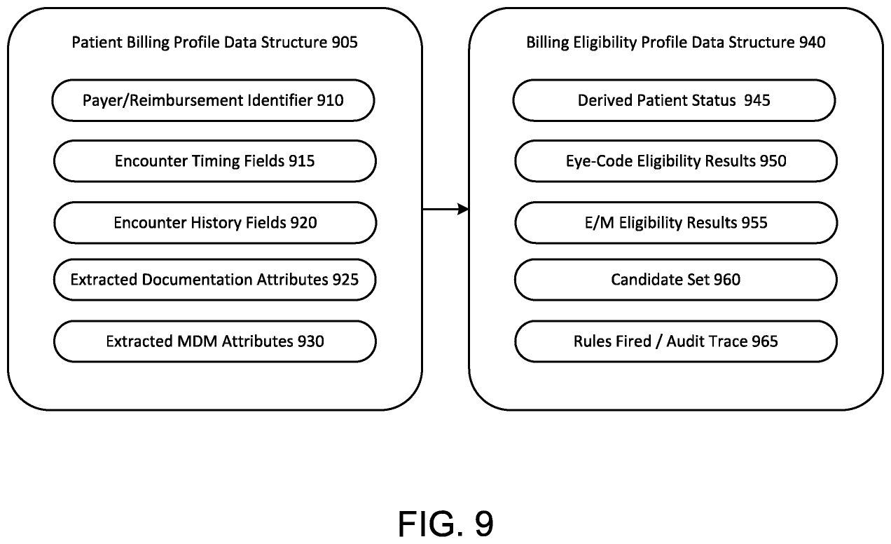

In some embodiments, the workflow may continue by generating a patient billing profile at step 710. For example, the patient billing profile may be generated as a structured data object or data structure, such as the patient billing profile data structure 905 described with reference to FIG. 9. For example, generating the patient billing profile may include constructing a structured data object that encodes discrete billing-relevant attributes including at least patient status, encounter timing information, and extracted encounter characteristics, the structured data object being configured for rule-based evaluation by an eligibility engine.

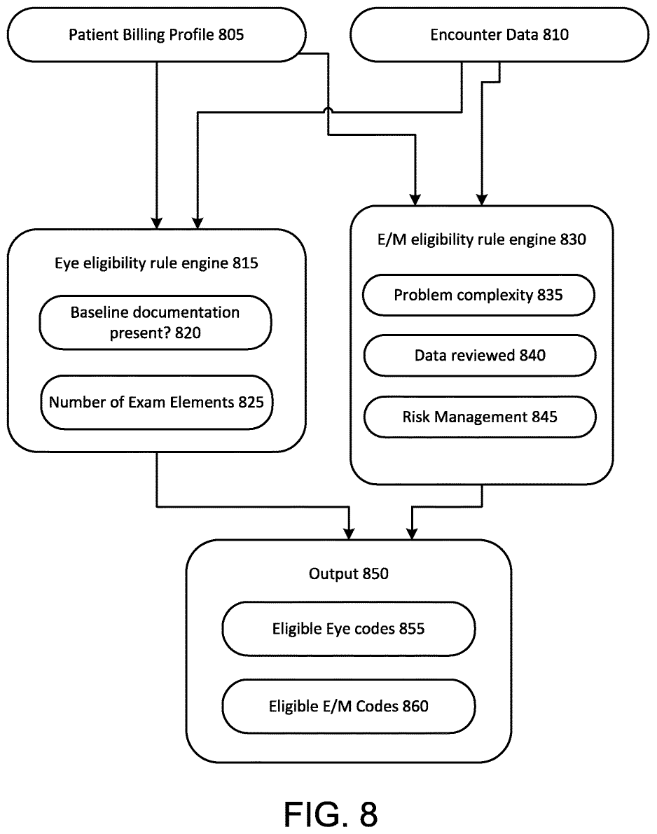

In some embodiments, generating the patient billing profile at step 710 may include encoding a reimbursement identifier corresponding to a payer associated with the encounter, encounter timing fields, encounter history fields, extracted documentation attributes, extracted medical-decision-making attributes, and/or other billing-relevant attributes into a machine-usable profile. For example, the patient billing profile may function as an intermediate data representation that reduces variability in source EMR formatting and permits subsequent rule-based evaluation to be performed against normalized fields rather than free-form source content. In some embodiments, the patient billing profile may therefore serve as a technical interface between EMR-derived encounter information and the eligibility engines described with reference to FIG. 8.

In some embodiments, the workflow may then proceed to applying eligibility rules at step 715. For example, the eligibility rules may be executed by the code selection engine 112, code selection engine 222, the hierarchical decision tree 312, and/or a rules engine implemented by instructions stored in storage 220 or memory 214. In some embodiments, the eligibility rules may operate on the patient billing profile and the obtained encounter data to determine whether particular encounter codes are eligible for the encounter. Unlike a generic reimbursement comparison, the eligibility rules may first constrain the code universe based on encounter-specific attributes before any reimbursement ranking is performed. In some embodiments, this constraint may reduce unnecessary reimbursement lookups, may reduce the risk of unsupported code selection, and may produce a more technically deterministic billing workflow.

In some embodiments, the eligibility rules may include determining patient status based on encounter history. For example, the system may evaluate encounter history and encounter timing information to determine whether the patient is new or established. In some embodiments, the determination may be based on whether the patient has no qualifying prior encounter, whether a threshold period has elapsed since a prior encounter, and/or whether the prior encounter history satisfies predefined status criteria. For example, the determination may be performed by comparing a current encounter date to stored prior-encounter timing information encoded in the patient billing profile. In some embodiments, the resulting patient status may be stored as a derived attribute, for example in the billing eligibility profile data structure 940 of FIG. 9, and may be used by multiple downstream rule subsets.

In some embodiments, after patient status has been determined, the workflow may proceed to applying subsets of eligibility rules at step 720. For example, a first subset of eligibility rules may be applied for ophthalmology-specific encounter code eligibility and a second subset of eligibility rules may be applied for evaluation-and-management encounter code eligibility. In some embodiments, the subsets may be evaluated independently, sequentially, in parallel, and/or in partially overlapping fashion. For example, the first subset may correspond to the Eye Code assessment at step 725 and the second subset may correspond to the E/M Code assessment at step 730. In some embodiments, the use of separate rule subsets may permit the system to process two different clinical coding frameworks using different technical criteria while still unifying results into a single candidate set for reimbursement analysis.

In some embodiments, the Eye Code assessment at step 725 may evaluate documentation-based criteria derived from encounter documentation. For example, the Eye Code assessment may evaluate whether baseline documentation components are present and whether a threshold number and/or type of ophthalmology exam elements are documented. In some embodiments, the Eye Code assessment may be performed using normalized documentation attributes extracted from chart notes, forms, checklists, and/or structured EMR fields, for example as produced by the data parser 302 of FIG. 3 and stored in the patient billing profile of FIG. 9. In some embodiments, the Eye Code assessment may produce a set of ophthalmology-specific encounter codes that satisfy applicable documentation criteria for the encounter.

In some embodiments, the E/M Code assessment at step 730 may evaluate medical-decision-making-based criteria derived from the encounter data. For example, the E/M Code assessment may evaluate problem complexity, data reviewed or analyzed, and/or risk of management. In some embodiments, the E/M Code assessment may operate on extracted medical-decision-making attributes encoded in the patient billing profile, such as indicators derived from chart review, orders, test interpretation activity, medication management, and/or other clinically relevant factors. For example, the E/M Code assessment may determine whether one or more MDM levels are satisfied based on a plurality-of-components analysis. In some embodiments, the E/M Code assessment may thereby produce a set of E/M encounter codes that satisfy applicable decision-making criteria for the encounter.

In some embodiments, the outputs of steps 725 and 730 may be merged to form a candidate set of documentation-eligible encounter codes at step 735. For example, the candidate set may include ophthalmology-specific encounter codes, evaluation-and-management encounter codes, or both, provided that each included code satisfies the applicable eligibility rules for the encounter. In some embodiments, step 735 may create a computationally constrained search space for downstream reimbursement processing by excluding ineligible codes before payer-specific reimbursement values are retrieved. For example, the candidate set may be stored in a billing eligibility profile data structure, such as the candidate set 960 of FIG. 9.

In some embodiments, the workflow may then proceed to obtaining payer-specific allowable reimbursement amounts at step 740. For example, the reimbursement calculator 118, 224 may use the reimbursement identifier from the patient billing profile to query payer-specific reimbursement data, such as reimbursement rates database 208 and/or insurance database 202, insurance database 122 described with reference to FIGS. 1 and 2. In some embodiments, payer-specific allowable reimbursement amounts may be retrieved only for codes included in the candidate set determined at step 735. This may improve efficiency by avoiding retrieval of reimbursement values for codes that are not eligible for the encounter and may provide a tighter technical linkage between the eligibility-determination stage and the reimbursement-ranking stage.

In some embodiments, the workflow may conclude by generating a reimbursement-based recommendation at step 745. For example, the reimbursement-based recommendation may be generated by ranking the candidate set of eligible encounter codes according to the obtained payer-specific allowable reimbursement amounts and causing presentation of at least one recommended encounter code within the electronic medical record. In some embodiments, the recommendation may include a ranked list, a top-ranked code, a highest allowable eligible code, and/or an electronically stored recommendation object usable by the EMR system. For example, the recommendation may be transmitted to the output generator 316 of FIG. 3 and/or the EMR integration engine 226 of FIG. 2 for display and/or storage. In some embodiments, because the recommendation is generated only after rule-based eligibility determination and constrained reimbursement retrieval, the workflow of FIG. 7 may provide a more technically specific and auditable billing recommendation process than a generalized reimbursement maximization process.

FIG. 8 illustrates exemplary eligibility rule evaluation engines configured to evaluate eligibility of ophthalmology-specific encounter codes and evaluation-and-management encounter codes using different subsets of criteria derived from encounter data. In some embodiments, FIG. 8 may represent a more detailed view of step 720, step 725, and step 730 of FIG. 7. For example, FIG. 8 may show how normalized attributes from the patient billing profile are utilized by separate rule engines to produce eligibility results for different code families.

In some embodiments, a patient billing profile 805 and encounter data 810 may be provided as inputs to the eligibility evaluation engines. For example, the patient billing profile 805 may correspond to the patient billing profile generated at step 710 of FIG. 7 and structured as shown in FIG. 9. In some embodiments, the encounter data 810 may include source clinical content and/or normalized attributes derived from source clinical content. For example, the encounter data 810 may include extracted chart information, exam findings, visit history, treatment-plan information, orders, note-derived features, and payer-linked context. In some embodiments, the patient billing profile 805 and encounter data 810 may be jointly supplied to multiple rule engines so that common inputs may be reused across different eligibility analyses.

In some embodiments, a first rule engine may include an Eye eligibility rule engine 815. For example, the Eye eligibility rule engine 815 may be configured to evaluate ophthalmology-specific encounter code eligibility based on documentation criteria. In some embodiments, the Eye eligibility rule engine 815 may first determine whether baseline documentation is present at 820. For example, baseline documentation may include a chief complaint, relevant history, general medical observation, and/or initiation of a diagnosis or treatment plan. In some embodiments, these components may be identified from structured EMR fields, parsed clinical notes, checklists, and/or other extracted documentation attributes. If baseline documentation is absent, the Eye eligibility rule engine 815 may determine that one or more Eye-code paths are not satisfied.

In some embodiments, the Eye eligibility rule engine 815 may further evaluate the number of exam elements at 825. For example, the Eye eligibility rule engine may determine whether a threshold number of ophthalmology exam elements has been documented, whether a subset of exam elements has been documented, and/or whether documentation satisfies criteria associated with different ophthalmology-specific encounter levels. In some embodiments, the exam elements may correspond to clinically relevant observations such as Visual acuity, Visual fields, Ocular motility, Conjunctiva, Adnexa, Pupil/iris, Cornea, Anterior chamber, Lens, Intraocular pressure, Optic disc, Retina, and/or other eye-exam content. For example, the Eye eligibility rule engine may compute documentation-element counts, may evaluate individual element-presence flags, and/or may classify the encounter into one or more documentation-supported Eye-code levels. In some embodiments, the output of the Eye eligibility rule engine 815 may be eligible Eye-codes 855, which may identify ophthalmology-specific encounter codes satisfying the documentation criteria for the encounter.

In some embodiments, a second rule engine may include an E/M eligibility rule engine 830. For example, the E/M eligibility rule engine 830 may be configured to evaluate evaluation-and-management encounter code eligibility based on medical decision-making criteria. In some embodiments, the E/M eligibility rule engine may evaluate problem complexity at 835, which may include attributes indicative of problem severity, chronicity, exacerbation, uncertainty, and/or complication level. The E/M eligibility rule engine may also evaluate data reviewed at 840, which may include indicators corresponding to reviewed notes, ordered tests, reviewed results, independently obtained history, independent interpretation activity, discussion with other providers, and/or other information-processing activity reflected in the encounter documentation. In some embodiments, the E/M eligibility rule engine may further evaluate risk of management at 845, which may include indicators associated with prescription management, procedure-related risk, follow-up decisions, and/or other management-related considerations.

In some embodiments, the E/M eligibility rule engine 830 may determine one or more E/M eligibility levels by evaluating the components 835, components 840, and components 845 individually and/or in combination. For example, the E/M eligibility rule engine 830 may determine whether a plurality of the components meet or exceed a given threshold level. In some embodiments, the E/M eligibility rule engine 830 may then output eligible E/M codes 860 corresponding to the determined E/M eligibility level or levels. As shown, the outputs 855 and 860 may be supplied to an output stage 850 that may forward the eligibility results to the workflow of FIG. 7, for example to support generation of the candidate set at step 735 and/or storage in the billing eligibility profile data structure of FIG. 9.

In some embodiments, FIG. 8 may therefore illustrate that different rule subsets may operate on different technical feature sets while still sharing a common patient billing profile and encounter dataset. For example, the Eye eligibility rule engine 815 may operate primarily on extracted documentation attributes, while the E/M eligibility rule engine 830 may operate primarily on extracted medical-decision-making attributes. This separation may improve modularity, may facilitate auditing of rule outputs, and may allow different rule families to be updated independently while preserving a unified downstream billing recommendation pipeline.

FIG. 9 illustrates exemplary structured data objects used by the billing code selection engine to store normalized encounter information, derived eligibility outputs, and intermediate billing results. In some embodiments, FIG. 9 may provide a structural view of the intermediate machine-usable data representations referenced in FIGS. 7 and 8. For example, the data structures of FIG. 9 may be created by the data parser 302 and algorithm processor 310 of FIG. 3 and may be consumed by the code selection engine 112 and reimbursement calculator 118 of FIG. 1.

In some embodiments, the system may generate a Patient Billing Profile Data Structure 905 from ophthalmology encounter data and payer information obtained from the electronic medical record. For example, the Patient Billing Profile Data Structure 905 may serve as a normalized intermediate object that organizes billing-relevant input information into discrete fields accessible to the eligibility rules. In some embodiments, the Patient Billing Profile Data Structure 905 may include a Payer/Reimbursement Identifier 910. For example, the Payer/Reimbursement Identifier 910 may identify a payer, plan, reimbursement category, locality, contract type, and/or other payer-linked parameter used by downstream reimbursement queries. In some embodiments, the Payer/Reimbursement Identifier 910 may provide a key used to retrieve payer-specific allowable reimbursement amounts after the candidate set has been determined, as shown at step 740 of FIG. 7.

In some embodiments, the Patient Billing Profile Data Structure 905 may further include Encounter Timing Fields 915 and Encounter History Fields 920. For example, the Encounter Timing Fields 915 may include a current encounter date, prior encounter dates, elapsed time values, and/or other temporal markers. The Encounter History Fields 920 may include indicators of prior encounters, historical visit counts, provider-history associations, and/or prior status-relevant facts. In some embodiments, the Encounter Timing Fields 915 and Encounter History Fields 920 may be used by the eligibility rules to determine whether the patient is new or established, for example as shown in FIG. 7. By storing these data as normalized fields rather than relying on ad hoc review of source notes, the system may improve the consistency and reproducibility of patient-status determinations.

In some embodiments, the Patient Billing Profile Data Structure 905 may also include Extracted Documentation Attributes 925. For example, the Extracted Documentation Attributes 925 may include structured indicators identifying whether baseline documentation components are present, whether particular exam elements have been documented, counts of documented exam elements, and/or other documentation-derived values usable by the Eye eligibility rule engine 815 of FIG. 8. In some embodiments, the Patient Billing Profile Data Structure 905 may further include Extracted MDM Attributes 930. For example, the Extracted MDM Attributes 930 may include indicators corresponding to problem complexity, data-review activity, risk-related management activity, and/or other features usable by the E/M eligibility rule engine 830 of FIG. 8. In some embodiments, the extracted attributes 925 and 930 may be generated from structured and/or unstructured source content, for example through parsing, normalization, keyword extraction, template parsing, rules-based feature extraction, natural language processing, and/or other computational preprocessing techniques.

In some embodiments, the system may generate a Billing Eligibility Profile Data Structure 940 by applying eligibility rules to the Patient Billing Profile Data Structure 905 and/or the encounter data. For example, the Billing Eligibility Profile Data Structure 940 may store derived outputs of the eligibility process and may act as an intermediate object between rule evaluation and reimbursement ranking. In some embodiments, the Billing Eligibility Profile Data Structure 940 may include a Derived Patient Status 945, such as a new or established patient status obtained from encounter-history evaluation. The Billing Eligibility Profile Data Structure 940 may also include Eye-code Eligibility Results 950 and E/M Eligibility Results 955, for example identifying which Eye-code and E/M-code pathways were satisfied and which encounter codes were determined to be eligible under each subset of rules.

In some embodiments, the Billing Eligibility Profile Data Structure 940 may further include a Candidate Set 960. For example, the Candidate Set 960 may represent a merged or unified set of encounter codes determined to be eligible for the encounter after application of the different rule subsets. In some embodiments, the Candidate Set 960 may define the population of codes for which reimbursement values are subsequently obtained. This may provide a technically constrained interface between the eligibility engine and the reimbursement engine because the reimbursement engine may query payer-specific allowables only for codes contained in the Candidate Set 960, rather than for a broader undifferentiated code set. In some embodiments, the Billing Eligibility Profile Data Structure 940 may optionally include a Rules Fired/Audit Trace 965, which may identify one or more rules, thresholds, conditions, or intermediate determinations contributing to the eligibility outputs. For example, the audit trace may permit downstream review, verification, quality assurance, compliance analysis, and/or explanation of how the candidate set was generated.

In some embodiments, the structured data objects of FIG. 9 may therefore provide a technically concrete framework for moving from heterogeneous EMR content to deterministic billing recommendations. For example, the Patient Billing Profile Data Structure 905 may normalize source data into rule-usable attributes, while the Billing Eligibility Profile Data Structure 940 may store derived eligibility outputs and constrained candidate sets used by downstream reimbursement ranking. In some embodiments, this separation between input-profile generation and eligibility-profile generation may improve modularity, traceability, and system maintainability while reducing reliance on manual interpretation of source documentation.

In some embodiments, the billing eligibility workflow described with reference to FIGS. 7-9 may be used to process different combinations of patient status, documentation-derived eligibility, medical-decision-making-derived eligibility, and payer-specific reimbursement data. For example, in one embodiment, the system may determine from encounter history that a patient is established, may determine from extracted documentation attributes that baseline ophthalmology documentation is present and that a higher documentation-supported Eye-code level is satisfied, and may further determine from extracted MDM attributes that a moderate E/M level is satisfied. In such an example, the system may generate Eye-code eligibility results identifying an ophthalmology-specific code, may generate E/M eligibility results identifying an E/M code, and may merge the results into a candidate set containing both codes. The system may then obtain payer-specific allowable reimbursement amounts for the codes in the candidate set and may generate a reimbursement-based recommendation by selecting, for example, a top-ranked or highest allowable eligible code for presentation within the electronic medical record.

In some embodiments, another example may involve an established patient for whom the extracted documentation attributes indicate that baseline ophthalmology documentation is present but only an intermediate documentation-supported Eye-code level is satisfied, while the extracted MDM attributes indicate that a lower E/M level is satisfied. In such an example, the system may determine a candidate set containing an intermediate Eye-code and a lower-level E/M code, may obtain payer-specific allowables for those candidate codes, and may generate the recommendation based on the relative payer-specific reimbursement amounts. In some embodiments, the recommendation may therefore differ across payers even where the candidate set remains unchanged, because the reimbursement identifier encoded in the patient billing profile may direct lookup of different payer-specific allowable values.

In some embodiments, another example may involve a new patient for whom the extracted documentation attributes indicate a more comprehensive Eye-code path and the extracted MDM attributes indicate a lower E/M level. In such an example, the patient-status determination may alter which specific encounter code is eligible under each path even though the same rule-engine architecture is used. In some embodiments, another example may involve a new patient for whom the Eye-code path yields an intermediate documentation-supported result while the E/M path yields a higher decision-making-supported result. The system may merge the eligibility results, constrain reimbursement lookup to the resulting candidate set, and rank the candidate set according to payer-specific allowables. In some embodiments, these examples demonstrate that the disclosed system does not merely compare reimbursement values in the abstract, but instead generates reimbursement-based recommendations only after applying structured eligibility rules to normalized encounter attributes and deriving a candidate set of eligible encounter codes from those rules.

In some embodiments, the billing eligibility workflow of FIGS. 7-9 includes guardrails and edge-case handling consistent with the eligibility-then-ranking sequence. For example, if encounter history is unavailable or insufficient to determine whether the patient is new or established, the system may (i) request additional encounter history from the electronic medical record and/or (ii) mark patient status as indeterminate and restrict eligibility evaluation to encounter codes whose eligibility does not depend on patient status, while recording the basis in an audit trace. In some embodiments, if baseline Eye documentation prerequisites are not satisfied, the Eye-code eligibility path may output no eligible Eye-codes while the E/M eligibility path remains eligible for evaluation; and if medical decision-making inputs are incomplete, the E/M eligibility path may output no eligible E/M codes or may output a reduced eligibility level based on available components. In some embodiments, if both paths output no eligible codes, the system may generate a null recommendation and present an exception indicator identifying unmet eligibility prerequisites. In some embodiments, when multiple eligible codes are produced by the Eye and E/M paths, the system builds a unified candidate set and obtains payer-specific allowable reimbursement amounts for the candidate set, and then selects and/or presents a top-ranked recommendation for the payer, subject to a compliance guardrail that any presented or selected recommendation is supported by documented encounter data.

In some embodiments, the billing workflow may further include compliance-oriented processing. For example, the system may maintain, within the billing eligibility profile data structure, a rules-fired or audit-trace representation indicating which eligibility conditions were satisfied and which candidate codes were included or excluded. In some embodiments, the audit-trace information may be used to support explanation of the recommendation, confirmation that the recommended code is supported by documented encounter data, post-hoc review of billing decisions, and/or validation of payer-specific ranking outcomes. For example, the presence of the audit trace may permit the recommendation pipeline to be both computationally constrained and operationally reviewable, thereby improving billing consistency while reducing unsupported code selection.

In some examples, the system may be integrated with a wide range of electronic medical record (EMR) systems. This integration capability may enable healthcare providers to incorporate the billing code selection functionality into their existing workflows. The system may utilize standardized APIs or custom integration modules to facilitate data exchange between the billing system and various EMR platforms.

Advanced features such as predictive analytics for billing trends may be incorporated into the system. These analytics may analyze historical billing data to identify patterns and predict future reimbursement rates or changes in coding requirements. This predictive capability may allow healthcare providers to proactively adjust their billing practices.

In some examples, the system may include a machine learning component that continuously improves code selection accuracy based on feedback. This adaptive learning capability may enable the system to refine its recommendations over time, taking into account factors such as variations in billing practices or changes in insurance policies.

The medical billing code selection system may be offered as a Software as a Service (SaaS) product to clinics. This delivery model may allow healthcare providers to access the system's functionality through a web-based interface without the need for extensive on-premises infrastructure. The SaaS approach may facilitate updates and maintenance of the system, thereby ensuring that users always have access to the latest features and coding information.

In some examples, the billing system may be licensed to EMR companies for integration into their existing software platforms. This licensing model may allow EMR providers to enhance their offerings with advanced billing selection capabilities. The integration may provide a seamless experience for healthcare providers, combining patient record management and billing selection within a single interface.

The system may include customizable rule sets that allow healthcare providers to tailor the code selection process to their specific needs. These rule sets may account for factors such as practice-specific billing preferences, insurance requirements, or specialty-specific coding guidelines. The ability to customize rules may enhance the system's flexibility across diverse healthcare settings.

In some examples, the system may incorporate natural language processing capabilities to analyze unstructured data from clinical notes or dictations. This feature may allow for more comprehensive analysis of patient encounters and may improve the accuracy of code recommendations.

The medical billing code selection system may include a reporting module that generates detailed analytics on billing performance. These reports may provide insights into reimbursement rates, denial patterns, and potential areas for improvement in coding practices. The reporting functionality may assist healthcare providers in making data-driven decisions to select their billing processes and financial performance.

The system may include a mobile application component that allows healthcare providers to access billing recommendations and perform code selection on-the-go. This mobile functionality may enhance the system's usability in clinical settings where providers may make billing decisions away from a desktop computer.

In some examples, the medical billing code selection system may incorporate blockchain technology for secure and transparent record-keeping of billing transactions. This feature may enhance the auditability of billing processes and may facilitate more efficient resolution of insurance claims.

The system may include a collaborative feature that allows multiple users within a healthcare organization to review and approve billing code selections. This collaborative approach may improve accuracy by enabling peer review of complex billing scenarios.

The medical billing code selection system may include a simulation mode that allows healthcare providers to model the financial impact of different coding strategies. This feature may enable practices to evaluate potential changes in their billing approach before implementation, supporting informed decision-making and strategic planning.

Although various embodiments have been described with reference to the figures, other embodiments are possible.

Although an exemplary system has been described with reference to FIGS. 1-6, other implementations may be deployed in other industrial, scientific, medical, commercial, and/or residential applications.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

In some aspects, the techniques described herein relate to a system including: a data store including a program of instructions; and, a processor operably coupled to the data store such that, when the processor executes the program of instructions, the processor causes operations to be performed to automatically generate a payer-specific reimbursement ranking for an encounter, the operations including: obtaining ophthalmology encounter data and payer information from an electronic medical record; generating, based on the ophthalmology encounter data and the payer information, a patient billing profile data structure that includes a reimbursement identifier corresponding to a payer associated with the encounter; applying a plurality of eligibility rules to the patient billing profile and the ophthalmology encounter data, including: determining a patient status indicating whether the patient is new or established, applying a first subset of eligibility rules configured to evaluate ophthalmology-specific encounter code eligibility based on documentation criteria, and applying a second subset of eligibility rules configured to evaluate evaluation-and-management encounter code eligibility based on medical decision-making criteria; based on the applied plurality of eligibility rules, determining a candidate set of eligible encounter codes including ophthalmology-specific codes and evaluation-and-management codes; obtaining, for each encounter code in the candidate set of eligible encounter codes, a payer-specific allowable reimbursement amount using the reimbursement identifier; and generating a reimbursement-based recommendation by ranking the candidate set of eligible encounter codes according to the obtained payer-specific allowable reimbursement amounts and causing presentation of at least one recommended encounter code within the electronic medical record.

In some aspects, the techniques described herein relate to a system, wherein generating the patient billing profile includes constructing a structured data object that encodes discrete billing-relevant attributes including at least patient status, encounter timing information, and extracted encounter characteristics, the structured data object being configured for rule-based evaluation by an eligibility engine.

In some aspects, the techniques described herein relate to a system, further including generating a billing eligibility profile data structure that identifies the candidate set of eligible encounter codes determined through application of the eligibility rules, the candidate set being constrained prior to any reimbursement evaluation.

In some aspects, the techniques described herein relate to a system, wherein the documentation criteria include evaluating whether a threshold number of ophthalmology exam elements are documented in the encounter data.

In some aspects, the techniques described herein relate to a system, wherein generating the reimbursement-based recommendation includes selecting, from the candidate set of eligible encounter codes, an encounter code having a highest payer-specific allowable reimbursement amount.

In some aspects, the techniques described herein relate to a computer-implemented method performed by at least one processor to automatically generate a payer-specific reimbursement ranking for an encounter, the method including: obtaining ophthalmology encounter data and payer information from an electronic medical record; generating, based on the ophthalmology encounter data and the payer information, a patient billing profile data structure that includes a reimbursement identifier corresponding to a payer associated with the encounter; applying a plurality of eligibility rules to the patient billing profile and the ophthalmology encounter data to determine eligibility of encounter codes for the encounter, including determining whether the patient is new or established; based on the applied plurality of eligibility rules, determining a candidate set of eligible encounter codes including ophthalmology-specific codes and evaluation-and-management codes; obtaining, for each encounter code in the candidate set of eligible encounter codes, a payer-specific allowable reimbursement amount using the reimbursement identifier; and generating a reimbursement-based recommendation by ranking the candidate set of eligible encounter codes according to the obtained payer-specific allowable reimbursement amounts and causing presentation of at least one recommended encounter code within the electronic medical record.

In some aspects, the techniques described herein relate to a computer-implemented method, wherein generating the patient billing profile includes constructing a structured data object that encodes discrete billing-relevant attributes including at least patient status, encounter timing information, and extracted encounter characteristics, the structured data object being configured for rule-based evaluation by an eligibility engine.

In some aspects, the techniques described herein relate to a computer-implemented method, further including generating a billing eligibility profile data structure that identifies the candidate set of eligible encounter codes determined through application of the eligibility rules, the candidate set being constrained prior to any reimbursement evaluation.

In some aspects, the techniques described herein relate to a computer-implemented method, further including applying a first subset of eligibility rules configured to evaluate ophthalmology-specific encounter code eligibility based on documentation criteria, and applying a second subset of eligibility rules configured to evaluate evaluation-and-management encounter code eligibility based on medical decision-making criteria.

In some aspects, the techniques described herein relate to a computer-implemented method, wherein payer-specific allowable reimbursement amounts are obtained only for encounter codes included in the candidate set of eligible encounter codes.

In some aspects, the techniques described herein relate to a computer-implemented method, wherein the documentation criteria include evaluating whether a threshold number of ophthalmology exam elements are documented in the encounter data.

In some aspects, the techniques described herein relate to a computer-implemented method, wherein applying the eligibility rules includes independently evaluating ophthalmology-specific encounter code eligibility criteria and evaluation-and-management encounter code eligibility criteria and merging the results into a unified candidate set of eligible encounter codes.

In some aspects, the techniques described herein relate to a computer-implemented method, wherein generating the reimbursement-based recommendation includes selecting, from the candidate set of eligible encounter codes, an encounter code having a highest payer-specific allowable reimbursement amount.

In some aspects, the techniques described herein relate to a computer program product including a program of instructions tangibly embodied on a non-transitory computer-readable medium that, when executed by one or more processors, cause the one or more processors to perform operations to automatically generate a payer-specific reimbursement ranking for an ophthalmology encounter, the operations including: obtaining ophthalmology encounter data and payer information from an electronic medical record; generating, based on the ophthalmology encounter data and the payer information, a patient billing profile data structure that includes a reimbursement identifier corresponding to a payer associated with the encounter; applying a plurality of eligibility rules to the patient billing profile and the ophthalmology encounter data, including: determining a patient status indicating whether the patient is new or established, applying a first subset of eligibility rules configured to evaluate ophthalmology-specific encounter code eligibility based on documentation criteria, and applying a second subset of eligibility rules configured to evaluate evaluation-and-management encounter code eligibility based on medical decision-making criteria; based on the applied plurality of eligibility rules, determining a candidate set of eligible encounter codes including ophthalmology-specific codes and evaluation-and-management codes; obtaining, for each encounter code in the candidate set of eligible encounter codes, a payer-specific allowable reimbursement amount using the reimbursement identifier; and generating a reimbursement-based recommendation by ranking the candidate set of eligible encounter codes according to the obtained payer-specific allowable reimbursement amounts and causing presentation of at least one recommended encounter code within the electronic medical record.

In some aspects, the techniques described herein relate to a computer program product, wherein generating the patient billing profile includes constructing a structured data object that encodes discrete billing-relevant attributes including at least patient status, encounter timing information, and extracted encounter characteristics, the structured data object being configured for rule-based evaluation by an eligibility engine.

In some aspects, the techniques described herein relate to a computer program product, further including generating a billing eligibility profile data structure that identifies the candidate set of eligible encounter codes determined through application of the eligibility rules, the candidate set being constrained prior to any reimbursement evaluation.

In some aspects, the techniques described herein relate to a computer program product, wherein payer-specific allowable reimbursement amounts are obtained only for encounter codes included in the candidate set of eligible encounter codes.

In some aspects, the techniques described herein relate to a computer program product, wherein the documentation criteria include evaluating whether a threshold number of ophthalmology exam elements are documented in the encounter data.

In some aspects, the techniques described herein relate to a computer program product, wherein applying the eligibility rules includes independently evaluating ophthalmology-specific encounter code eligibility criteria and evaluation-and-management encounter code eligibility criteria and merging the results into a unified candidate set of eligible encounter codes.

In some aspects, the techniques described herein relate to a computer program product, wherein generating the reimbursement-based recommendation includes selecting, from the candidate set of eligible encounter codes, an encounter code having a highest payer-specific allowable reimbursement amount.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A system comprising:
a data store comprising a program of instructions; and,
a processor operably coupled to the data store such that, when the processor executes the program of instructions, the processor causes operations to be performed to automatically generate a payer-specific reimbursement ranking for an encounter, the operations comprising:
obtaining ophthalmology encounter data and payer information from an electronic medical record;
generating, based on the ophthalmology encounter data and the payer information, a patient billing profile data structure that includes a reimbursement identifier corresponding to a payer associated with the encounter;
applying a plurality of eligibility rules to the patient billing profile and the ophthalmology encounter data, comprising:
determining a patient status indicating whether the patient is new or established,
applying a first subset of eligibility rules configured to evaluate ophthalmology-specific encounter code eligibility based on documentation criteria, and
applying a second subset of eligibility rules configured to evaluate evaluation-and-management encounter code eligibility based on medical decision-making criteria;
based on the applied plurality of eligibility rules, determining a candidate set of eligible encounter codes including ophthalmology-specific codes and evaluation-and-management codes;
obtaining, for each encounter code in the candidate set of eligible encounter codes, a payer-specific allowable reimbursement amount using the reimbursement identifier; and
generating a reimbursement-based recommendation by ranking the candidate set of eligible encounter codes according to the obtained payer-specific allowable reimbursement amounts and causing presentation of at least one recommended encounter code within the electronic medical record.

2. The system of claim 1, wherein generating the patient billing profile comprises constructing a structured data object that encodes discrete billing-relevant attributes including at least patient status, encounter timing information, and extracted encounter characteristics, the structured data object being configured for rule-based evaluation by an eligibility engine.

3. The system of claim 1, further comprising generating a billing eligibility profile data structure that identifies the candidate set of eligible encounter codes determined through application of the eligibility rules, the candidate set being constrained prior to any reimbursement evaluation.

4. The system of claim 1, wherein the documentation criteria include evaluating whether a threshold number of ophthalmology exam elements are documented in the encounter data.

5. The system of claim 1, wherein generating the reimbursement-based recommendation comprises selecting, from the candidate set of eligible encounter codes, an encounter code having a highest payer-specific allowable reimbursement amount.

6. A computer-implemented method performed by at least one processor to automatically generate a payer-specific reimbursement ranking for an encounter, the method comprising:
obtaining ophthalmology encounter data and payer information from an electronic medical record;
generating, based on the ophthalmology encounter data and the payer information, a patient billing profile data structure that includes a reimbursement identifier corresponding to a payer associated with the encounter;
applying a plurality of eligibility rules to the patient billing profile and the ophthalmology encounter data to determine eligibility of encounter codes for the encounter, including determining whether the patient is new or established;
based on the applied plurality of eligibility rules, determining a candidate set of eligible encounter codes including ophthalmology-specific codes and evaluation-and-management codes;
obtaining, for each encounter code in the candidate set of eligible encounter codes, a payer-specific allowable reimbursement amount using the reimbursement identifier; and
generating a reimbursement-based recommendation by ranking the candidate set of eligible encounter codes according to the obtained payer-specific allowable reimbursement amounts and causing presentation of at least one recommended encounter code within the electronic medical record.

7. The computer-implemented method of claim 6, wherein generating the patient billing profile comprises constructing a structured data object that encodes discrete billing-relevant attributes including at least patient status, encounter timing information, and extracted encounter characteristics, the structured data object being configured for rule-based evaluation by an eligibility engine.

8. The computer-implemented method of claim 6, further comprising generating a billing eligibility profile data structure that identifies the candidate set of eligible encounter codes determined through application of the eligibility rules, the candidate set being constrained prior to any reimbursement evaluation.

9. The computer-implemented method of claim 6, further comprising applying a first subset of eligibility rules configured to evaluate ophthalmology-specific encounter code eligibility based on documentation criteria, and applying a second subset of eligibility rules configured to evaluate evaluation-and-management encounter code eligibility based on medical decision-making criteria.

10. The computer-implemented method of claim 9, wherein payer-specific allowable reimbursement amounts are obtained only for encounter codes included in the candidate set of eligible encounter codes.

11. The computer-implemented method of claim 9, wherein the documentation criteria include evaluating whether a threshold number of ophthalmology exam elements are documented in the encounter data.

12. The computer-implemented method of claim 6, wherein applying the eligibility rules comprises independently evaluating ophthalmology-specific encounter code eligibility criteria and evaluation-and-management encounter code eligibility criteria and merging the results into a unified candidate set of eligible encounter codes.

13. The computer-implemented method of claim 6, wherein generating the reimbursement-based recommendation comprises selecting, from the candidate set of eligible encounter codes, an encounter code having a highest payer-specific allowable reimbursement amount.

14. A computer program product comprising a program of instructions tangibly embodied on a non-transitory computer-readable medium that, when executed by one or more processors, cause the one or more processors to perform operations to automatically generate a payer-specific reimbursement ranking for an ophthalmology encounter, the operations comprising:
obtaining ophthalmology encounter data and payer information from an electronic medical record;
generating, based on the ophthalmology encounter data and the payer information, a patient billing profile data structure that includes a reimbursement identifier corresponding to a payer associated with the encounter;
applying a plurality of eligibility rules to the patient billing profile and the ophthalmology encounter data, comprising:
determining a patient status indicating whether the patient is new or established,
applying a first subset of eligibility rules configured to evaluate ophthalmology-specific encounter code eligibility based on documentation criteria, and
applying a second subset of eligibility rules configured to evaluate evaluation-and-management encounter code eligibility based on medical decision-making criteria;
based on the applied plurality of eligibility rules, determining a candidate set of eligible encounter codes including ophthalmology-specific codes and evaluation-and-management codes;
obtaining, for each encounter code in the candidate set of eligible encounter codes, a payer-specific allowable reimbursement amount using the reimbursement identifier; and
generating a reimbursement-based recommendation by ranking the candidate set of eligible encounter codes according to the obtained payer-specific allowable reimbursement amounts and causing presentation of at least one recommended encounter code within the electronic medical record.

15. The computer program product of claim 14, wherein generating the patient billing profile comprises constructing a structured data object that encodes discrete billing-relevant attributes including at least patient status, encounter timing information, and extracted encounter characteristics, the structured data object being configured for rule-based evaluation by an eligibility engine.

16. The computer program product of claim 14, further comprising generating a billing eligibility profile data structure that identifies the candidate set of eligible encounter codes determined through application of the eligibility rules, the candidate set being constrained prior to any reimbursement evaluation.

17. The computer program product of claim 14, wherein payer-specific allowable reimbursement amounts are obtained only for encounter codes included in the candidate set of eligible encounter codes.

18. The computer program product of claim 14, wherein the documentation criteria include evaluating whether a threshold number of ophthalmology exam elements are documented in the encounter data.

19. The computer program product of claim 14, wherein applying the eligibility rules comprises independently evaluating ophthalmology-specific encounter code eligibility criteria and evaluation-and-management encounter code eligibility criteria and merging the results into a unified candidate set of eligible encounter codes.

20. The computer program product of claim 14, wherein generating the reimbursement-based recommendation comprises selecting, from the candidate set of eligible encounter codes, an encounter code having a highest payer-specific allowable reimbursement amount.
